(19) United States
(12) Patent Application Publication
(10) Pub. No.: US 2016/0317228 A1
(43) Pub. Date: Nov. 3, 2016

(54) METHODS AND SYSTEMS FOR HIGH SPEED LASER SURGERY

(71) Applicant: IMRA America, Inc., Ann Arbor, MI (US)

(72) Inventors: Martin E. Fermann, Dexter, MI (US); Christopher J. Hensley, Ann Arbor, MI (US)

(21) Appl. No.: 15/208,432

(22) Filed: Jul. 12, 2016

Related U.S. Application Data
(63) Continuation of application No. PCT/US2015/011802, filed on Jan. 16, 2015.
(60) Provisional application No. 61/930,405, filed on Jan. 22, 2014.

(51) Int. Cl. A61B 18/22; A61B 5/00

(52) U.S. Cl. CPC A61B 18/22; A61B 5/0066; A61B 5/0059; A61B 2018/2227; A61B 2018/2222; A61B 2018/2211; A61B 2018/209

(57) ABSTRACT

The present disclosure relates to a methods and systems for high speed laser surgery. In some implementations, the combination of mid-infrared (mid-IR) laser radiation with micro-scanning technology allows for large tissue ablation rates with minimal thermally affected zones, where micro-scanning distributes the heat generated by laser surgery over a large tissue area. Micro-scanning technology is compatible with hollow core fiber technology which can be implemented to deliver near diffraction limited mid-IR laser beams into the vicinity of the target area. Micro-scanning technology is compatible with hand tools for direct replacement of mechanical surgical tools such as scalpels as well as robotic surgery. Micro-scanning technology is also compatible with endoscopic beam delivery and can be combined with endoscopic tissue analysis. Tissue analysis can be performed with optical imaging technology as well as other analytical tools such as mass spectrometers.

### CROSS-REFERENCE TO RELATED APPLICATIONS

This application is a continuation of international application no. PCT/US2015/011802, filed Jan. 16, 2015, entitled “METHODS AND SYSTEMS FOR HIGH SPEED LASER SURGERY,” which claims the benefit of priority to U.S. Patent Application No. 61/930,405, filed Jan. 22, 2014, entitled “METHODS AND SYSTEMS FOR HIGH SPEED LASER SURGERY;” each of which is hereby incorporated by reference herein in its entirety.

### BACKGROUND

1. Field

The present disclosure relates generally to laser surgery systems and methods for increasing the speed of laser surgery.

2. Description of Related Art

Lasers are well established in many medical applications as an essential tool for an ever increasing number of therapies and also to treat an ever increasing number of diseases. Though laser applications are quite varied, it is possible to at least find a couple of overarching design principles that govern a large subset of applications, especially with regards to surgery and in particular in precision surgery.

### SUMMARY

A continuing need exists for technical advancements which support increased speed of laser surgery while avoiding unacceptable collateral damage to tissue. It is preferred to reduce the footprint of laser surgery systems and provide flexible tools for both robotic and manual surgery.

In one aspect the present disclosure features new system architectures for efficient and rapid laser surgery with minimal thermally affected damage zones. Any type of surgery can be performed, of particular interest is surgery performed under thermal confinement of the heat generated by the laser. Also laser surgery with the benefit of stress confinement can be carried out. The surgery can be executed with optical beam delivery systems, such as hollow core fibers acting as transfer fibers which efficiently transfer an input beam along the fiber and provide an output beam for laser surgery. The transfer fiber is configured to receive pulsed radiation from a source and transfer the pulsed radiation to an output of said transfer fiber.

The pulse width utilized at the input to the transfer fiber can be in a range from around 10 ps to a few μs, and up to about 10 μs. In some implementations pulses with a width in the range from 100 fs-10 ps can be used. Pulse bursts which include a series of short pulses can also be implemented to increase the overall laser energy or average power transmitted.

The transfer fiber can be made of a material with a high damage threshold and high transmission in the 1-4 μm wavelength window, such as silica or germania glass.

Several types of hollow core germania and silica fibers can be used, such as micro-structured fibers, photonic crystal fibers (PCFs), Kagome fiber or hypocycloid fibers. These fibers can be manufactured by extrusion and can also be tapered.

To allow tight bending diameters, hollow core photonic crystal fibers can be employed. To enable the transmission of high pulse energies without damage, pulse bursts can be used. Pulse bursts are preferably provided with an overall temporal length within the thermal confinement time.

To increase the speed of laser surgery the average laser power that is transferred to the sample is maximized. This can be accomplished with reasonably sized laser technology via an increase of the laser repetition rate.

Heat accumulation and excessive thermal damage is avoided by rapid scanning over the sample area. Rapid scanning can be carried out close to the sample by using appropriate fiber transfer systems with integrated micro-scanning technology. Micro-scanning laser surgery is further compatible with beam delivery via hollow core fibers and any laser technology, provided the laser light is efficiently transmitted through the fiber.

A variety of laser transfer or beam delivery systems can be implemented for micro-scanning. Of particular interest are micro-scanning devices that enable resonant mechanical excitation of a fiber cantilever tip. In particular, in some embodiments the fiber tip may be configured with a free-portion near the fiber output end, without the free-portion mounted for mechanical support. In such an arrangement the free-portion will rapidly vibrate (e.g.: dither) which supports high speed operation. To improve the speed of the fiber tip, hollow core fibers with a limited outside diameter in the range from 100-200 μm can be implemented. The scanning cantilever tip can be combined with other scanning modalities such as scanning mirrors, prisms, gratings or micro-electromechanical mirrors or MEMS.

These laser transfer or beam delivery systems can be handheld to serve as replacements for actual mechanical surgical devices.

Also rapid laser scanning can be performed with conventional scanning technology. Beam guidance systems can further be implemented to identify the scanning area or to help with appropriate focusing. For example low power visible laser light can be transmitted along with the high power laser source along the hollow fiber to guide with surgery. In addition laser beams designed to perform different functionalities such as photo-coagulation can be transmitted along the hollow fiber. Beam pointing lasers and photo-coagulation lasers can also be transmitted using separate waveguides adjacent to the transfer fiber.

Optical analytical measurement devices can be further provided with the surgery system to analyze the target area. For example optical coherence tomography (OCT), infrared (IR) microscopy, multi-photon microscopy, endoscopy, two-dimensional or three-dimensional (2D or 3D) video cameras as well as thermal imaging can be used to guide in surgery.

Adaptive feedback can further be provided in such surgery systems via attachment of appropriate analytical tools such as a mass or optical spectrometer. For example certain molecules generated during surgery can be transmitted through a suction tube and analyzed during surgery. Other feedback mechanisms based on optical coherence tomography, fluorescence or infrared microscopy or multi-photon microscopy can also be implemented. Such imaging modalities can provide feedback during surgery to measure the extent of diseased tissue. Also other deeper penetrating laser light can be provided during laser surgery to localize precious biological parts such as nerves that need to be left unharmed during surgery.

The systems discussed here are compatible with almost any precision laser surgery. The systems can also be combined with robotic laser surgery where an environmental interface is moved by a robot rather than a surgeon. The systems can be configured as laser endoscopes, where a transfer fiber transmits high power laser energy as well as low power light implemented for imaging. Scattered light from the surgical target area can be directed back for further analysis using the transfer fiber as well as additional fibers.

A variety of laser architectures can be used to generate the laser pulses. An example of a preferred range is a laser wavelength in the range from 1.0-2.5 μm. Examples of such laser architectures are Yb, Er, Tm or Ho fiber lasers. Other alternatives are solid-state Er:YAG, Er:YSGG, Cr:ZnSe, Tm:YAG, Ho:YAG, Nd:YAG or Yb:YAG based laser systems. These laser systems can be combined with frequency shifting nonlinear crystals to access certain preferred wavelength ranges. In at least one preferred implementation laser pulses are generated in the 1.1-3. 5 μm wavelength range, and transmitted through a transfer fiber for laser surgery. For example, laser pulses with wavelengths in the mid-infrared (mid-IR) can be utilized.

Optical parametric generation or amplification in nonlinear crystals can be used for frequency shifting. Also optical parametric oscillators can be used for the same purpose. Frequency shifting via optical parametric oscillators can be efficiently performed using pulse bursts.

Alternatively, pulsed high power mid-IR generation for laser surgery can be based on efficient Raman shifting or four-wave mixing in hollow core fibers. The Raman shifting fiber can further be used as a transfer fiber, where the light input at one end of the fiber at one frequency is transferred to a distal second fiber end producing a frequency shifted output at a different frequency. Residual light at the first frequency can be blocked by a bulk optical element such as a dielectric filter or continuously along the fiber via fiber tapering. More than one fiber section can be used. A variety of Raman active gases can be used for Raman shifting, these comprise for example H2, D2, N2, and/or methane. The pressure of the Raman active gases in the hollow core fiber can be between 1-100 atm (1 atm=101,325 Pa).

The high power mid-IR light sources described here can be used for a variety of applications and not only in the biomedical realm, for example the high power mid-IR light can also be used in laser machining and micro-machining as well as laser deposition applications in the manufacturing of polymers. The compactness of these sources is further attractive in mass spectrometry application, where the mid-IR light can be used for laser desorption as well as laser ionization in the presence of adequate solvents or matrices.

### BRIEF DESCRIPTION OF THE DRAWINGS

FIG. 1 schematically illustrates components of conventional laser surgery systems.

FIG. 2A schematically illustrates a laser surgery system according to an embodiment of this disclosure.

FIG. 2B schematically illustrates a micro-scanning laser surgery system according to an embodiment of this disclosure.

FIG. 2C schematically illustrates an embodiment of a micro-scanning laser surgery system.

FIG. 3 schematically illustrates an embodiment of a laser system for generating high power mid- IR pulse bursts.

FIG. 4A schematically illustrates an example arrangement for coherently combining two pulses from two fiber amplifiers.

FIG. 4B schematically illustrates an example arrangement for coherently combining four pulses from two fiber amplifiers.

FIG. 5 schematically illustrates an embodiment of a micro-surgery system with integrated imaging modalities.

FIG. 6 schematically illustrates an embodiment of a laser surgery system which incorporates an exemplary hollow-core photonic bandgap and/or holey fiber(s) which may be implemented as transfer fiber(s), and provide for optional wavelength shifting.

FIG. 6A shows a cross sectional view of an example of a hollow core fiber.

The figures depict various embodiments of the present disclosure for purposes of illustration and are not intended to be limiting. Alternative embodiments of the systems and methods illustrated herein may be employed without departing from the principles described herein. Additional figures schematically illustrating additional embodiments of the disclosure are included in the various patents, patent publications, and patent applications incorporated by reference herein. Reference will now be made in detail to several embodiments, examples of which are illustrated in the accompanying figures. It is noted that wherever practicable similar or like reference numbers may be used in the figures and may indicate similar or like functionality.

### DETAILED DESCRIPTION

### Overview

The following patents, published patent applications, and non-patent publications are pertinent to the present disclosure.

U.S. Pat. No. 3,467,098, ‘Flexible conduit for laser surgery’, W. A. Ayres et al.

U.S. Pat. No. 5,782,822, ‘Method and apparatus for removing corneal tissue with infrared laser radiation’, Telfair et al.

U.S. Pat. No. 5,957,915 ‘Hand-held laser scanner’, D. Trost et al.

U.S. Pat. No. 6,552,301, ‘Burst-Ultrafast laser Machining Method’, P. Hermann et al.

U.S. Pat. No. 7,167,622, ‘Photonic crystal fibers and medical systems including photonic crystal fibers’, Temelkuran et al.

U.S. Pat. No. 7,656,578, ‘Microchip-Yb fiber hybrid optical amplifier for micro-machining and marking’, Galvanauskas et al.

U.S. Pat. No. 7,414,780, ‘All-fiber chirped pulse amplification systems’, Fermann et al.,

U.S. Pat. No. 7,486,705, Temtosecond laser processing system with process parameters, controls and feedback', L. Shah et al.

U.S. Pat. No. 8,029,501, ‘Laser selective cutting by impulsive heat deposition in the IR wavelength range for direct-drive ablation’, Miller et al.

U.S. Pat. No. 8,500,724, ‘Method and apparatus for patterned plasma-mediated laser trephination of the lens capsule and three dimensional phaco-segmentation’, Blumenkranz et al.

U.S. Pat. No. 8,074,661, ‘Method and apparatus for laser tissue ablation’, Hutson et al.

U.S. Pat. No. 8,285,099, ‘Large core holey fibers’, Dong et al.

U.S. Pat. No. 8,478,097, ‘Wide bandwidth, low loss, photonic bandgap fibers’, Dong et al.

U.S. Pat. No. 8,553,337, ‘Multi-path, multi-magnification, non-confocal fluorescence emission endoscopy apparatus and methods’, Webb et al.

L. Giniunas et al. ‘Endoscopy with optical sectioning capability’, App. Opt., vol. 32, pp. 2888 (1993).

U.S. Patent Application Pub. No. 2010/0286674, ‘Systems, devices and methods for imaging and surgery’, Ben-Yakar et al.

U.S. Patent Application Pub. No. 2012/0302828, ‘Apparatus, system and method for providing laser steering and focusing for incision, excision and ablation of tissue in minimally-invasive surgery’, Toledo-Crow et al.

B. Beaudou et al., ‘Matched cascade of bandgap-shift and frequency-conversion using stimulated Raman scattering in a tapered hollow-core photonic crystal fibre’, Opt. Expr., vol. 18, pp. 12381 (2010).

D. Dickensheets et al., ‘A scanned Optical Fiber Confocal Microscope’, SPIE, vol. 2184, pp. 39 (1994).

G. S. Edwards, ‘Mechanisms for soft-tissue ablation and the development’, Laser & Photonics Reviews, vol. 3, No. 6, pp. 545-555 (2009).

K. Franjic et al., ‘Laser selective cutting of biological tissues by impulsive heat deposition through ultrafast vibrational excitations’, Opt. Expr., vol. 17, pp. 22937 (2009).

Mark A. Mackanos, ‘The effect of pulse structure on soft tissue laser ablation at mid-infrared wavelengths’, Ph.D. Thesis, Vanderbilt University (2004).

MIRSURG, Grant Agreement number: 224042, final report: ‘Mid-Infrared Solid-State Laser Systems for Minimally Invasive Surgery’, coordinated by V. Petrov, Nov. 2011.

A. Ulrich et al., ‘Silica hollow core micro structured fibres for mid-infrared surgical applications’, Journal of Non-Crystalline Solids, vol. 377, pp. 236-239 (2013).

Vogel et al., ‘Pulsed Laser Ablation of Soft Biological Tissues’ in Optical Thermal Response of Laser Irradiated Tissue (2011).

Wei et al., ‘Fiber laser pumped high power mid-infrared laser with picosecond pulse bunch output’, Opt. Expr., vol. 21, pp. 25364 (2013).

The following U.S. patents are hereby incorporated by reference herein in their entirety: U.S. Pat. No. 7,656,578, ('578), ‘Microchip-Yb fiber hybrid optical amplifier for micro-machining and marking’, Galvanauskas et al.; U.S. Pat. No. 7,414,780, ('780), ‘All-fiber chirped pulse amplification systems’, Fermann et al; U.S. Pat. No. 7,486,705, ('705), Temtosecond laser processing system with process parameters, controls and feedback', Shah et al.

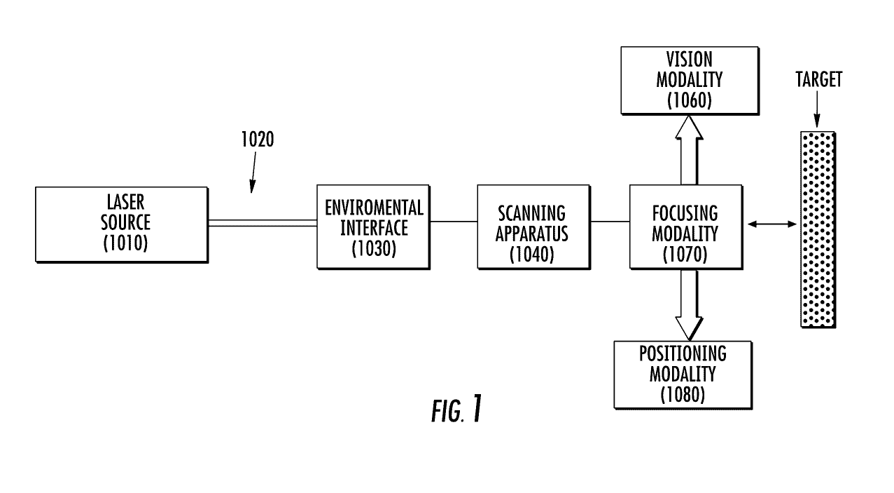

Various components of a conventional laser surgery system are shown in FIG. 1. The laser surgery system includes at least one laser source 1010. The laser beam output from the source is then transmitted along or by a beam delivery system 1020. Many different laser architectures are well known in the art. One such laser example can be based on fiber laser technology as disclosed in U.S. Pat. No. 7,656,578, ‘Microchip-Yb fiber hybrid optical amplifier for micro-machining and marking’, Galvanauskas et al.

Beam delivery system(s) 1020 can include, for example, articulated arms, conduits (see U.S. Pat. No. 3,467,098: ‘Flexible conduit for laser surgery’, W. A. Ayres et al.) or optical waveguides such as fibers. Basic beam delivery with optical waveguides has been for example discussed in the following patents: U.S. Pat. No. 8,074,661 ‘Method and apparatus for laser tissue ablation’ to Hutson et al.; U.S. Pat. No. 5,782,822 ‘Method and apparatus for removing corneal tissue with infrared laser radiation’ to Telfair et al. and U.S. Pat. No. 8,029,501 ‘Laser selective cutting by impulsive heat deposition in the IR wavelength range for direct-drive ablation’ to Miller et al. ('501).

The delivery systems may be terminated in an environmental interface 1030 which can, for example, contain a high power beam as well as a visible pointing beam in a handheld device for manual operation with a fiber attached. Another example can be the end of an articulated arm for semiautomatic or robotic surgery. Both a visible pointing beam and the high power surgery beam can also be delivered via the articulated arm. The end of the articulated arm can also hold a transfer fiber for optically contained light transfer from the laser to the surgical area.

Oftentimes the laser beam is also transmitted through an optical scanning arrangement 1040 and is finally directed to the target with a focusing arrangement, focusing modality 1070. Such arrangements have been known from confocal microscopy for a long time, see L. Giniunas et al. ‘Endoscopy with optical sectioning capability’, App. Opt., vol. 32, pp. 2888 (1993), where optical scanning was achieved by moving a fiber tip in front of a focusing lens. Alternatively, also a small focusing lens can be moved rapidly in front of a fiber tip to enable rapid scanning, as described in D. Dickensheets et al., ‘A scanned Optical Fiber Confocal Microscope’, SPIE, vol. 2184, pp. 39 (1994)

A vision modality 1060 (having one or more vision modalities) may be provided that enable the surgeon to inspect the target area, for example surgical microscopes with display technology can be used. In precision surgery endoscopes, for example, coherent fiber arrays are popular as a vision modality. In addition positioning modalities 1080 are oftentimes provided that help with the beam movement across the target. Such positioning modalities can be combined with medical endoscopes as discussed in U.S. Pat. No. 7,167,622, ‘Photonic crystal fibers and medical systems including photonic crystal fibers’ to Temelkuran et al. '622 also describes cooling modalities (not shown in FIG. 1) that for example cool a delivery fiber when high power laser light is being used. Different system implementations of these various elements are well known in the state of the art.

For many applications various components of laser surgery have been combined into single packaged devices. For example fiber beam delivery has also been combined with medical endoscopes as described in '622. In another example an environmental interface, beam scanning, and beam delivery has been combined into a single endoscopic device as disclosed in U.S. Pat. No. 8,553,337: ‘Multi-path, multi-magnification, non-confocal fluorescence emission endoscopy apparatus and methods’ to Webb et al. and in U.S. Patent Application Pub. No. 2010/0286674: ‘Systems, devices and methods for imaging and surgery’ to Ben-Yakar et al. ('674).

A handheld environmental interface with a beam scanner has been described in U.S. Pat. No. 5,957,915 ‘Hand-held laser scanner’ to D. Trost et al. A more compact laser scanning system for laser surgery has been described in US patent application Pub. No. 2012/0302828, ‘Apparatus, system and method for providing laser steering and focusing for incision, excision and ablation of tissue in minimally-invasive surgery’ to Toledo-Crow et al ('828). Such systems can also be combined with fiber optic beam delivery.

Vision modalities have been further combined with laser surgery for example in ‘828 and also in U.S. Pat. No. 8,500,724, ‘Method and apparatus for patterned plasma-mediated laser trephination of the lens capsule and three dimensional phacosegmentation’ to Blumenkranz et al., where optical coherence tomography was used as a vision modality.

However, current laser surgery systems suffer from some deficiencies. For example, when using CO₂ lasers in surgery, deep heat affected damage zones are generated that can typically induce large areas of necrotic cells, produce inflammation and delay healing.

Beam delivery systems based on articulated arms are relatively large and offer limited degrees of freedom for beam movement. Other beam delivery systems are oftentimes based on multimode fibers which may complicate focusability of the laser beam on the target area. Yet other beam delivery systems use fibers with alternating layers of dielectrics which may need cooling for their operation at high power as discussed in '622.

The optical power levels and repetition rates (for systems relying on short pulses) of current surgery lasers are oftentimes severely restricted due to either limitations in the actual laser material or thermal damage in the target material. Also the laser pulse widths implemented in surgery may not be optimal.

Scanning modalities are oftentimes relatively large and not easily compatible with environmental interfaces such as handheld beam pointers, also scan speed can be limited.

Oftentimes vision modalities require an extra component such as an endoscope to be positioned in the vicinity of the surgical area which poses problems when accurately assessing the performance of an actual surgery.

Some of the prior art systems have addressed some of these deficiencies, while not dealing with others.

For precision surgery, pulsed mid-IR radiation has been shown to have some benefits as is well known in the state of the art. Mid-IR radiation allows resonant absorption of water or proteins such as collagen at wavelengths near 3 and 6.5 μm respectively. With mid-IR radiation the ablation threshold becomes mainly a parameter of the total energy density absorbed in the tissue and is only weakly dependent on peak power as is well known in the state of the art. Nevertheless there is a large variation of laser fluences that have been used even for resonant laser absorption, depending on the sought application. Mostly, the applied laser fluences are in the range of 0.1 to 10 J/cm². However, other fluences may also be used; for example, with laser pulses in the microsecond (μs)—millisecond (ms) range, laser fluences up to at least about 100 J/cm² have also been implemented.

Specifically, mid-IR surgery performed with free electron lasers (FELs), has shown some encouraging results, but the size and cost of these systems has prevented their widespread use. More recently, pulsed mid-IR solid-state lasers have been used for laser surgery, but the rate of ablation is still a limitation that presents a high barrier for actual system implementation (G. S. Edwards, ‘Mechanisms for soft-tissue ablation and the development’, Laser & Photonics Reviews, vol. 3, No. 6, pp. 545-555 (2009) and also MIRSURG, Grant Agreement number: 224042, final report: ‘Mid-Infrared Solid-State Laser Systems for Minimally Invasive Surgery’, coordinated by V. Petrov, November 2011.

A key motivation for the use of pulsed mid IR laser radiation in surgery is to enable the application of laser radiation under the condition of thermal confinement, i.e. to minimize heat accumulation during the ablation process or to use laser exposures shorter than a characteristic thermal diffusion time of heat out of the heated zone. Operating under the condition of thermal confinement minimizes collateral damage in tissue ablation, which preferably is minimized to minimize tissue inflammation. Thermal confinement can be ensured for single pulses when using pulse widths shorter than a few μs near the water absorption peak near 3000 nm in tissue. However, there has been limited hope so far that efficient surgical applications could be demonstrated when operating short pulse lasers in the thermal confinement regime.

For example in ‘501 it was estimated that the time T between individual ablating pulses should be T>(2R)²/6D, where R is the laser spot diameter and D the thermal diffusivity of the target material. With the suggested value of D−10⁻⁷ m²/s and 2R in the range from 10-100 μm, we obtain the range for T: 170 μs<T<17 ms or a permissible repetition rate between 60 Hz -6 kHz with the higher repetition rates suggested for smaller spot sizes. Spot diameters smaller than around 25 μm are generally not used for non-ophthalmic surgeries unless surgical intervention is sought at the cellular level. Hence repetition rates greater than about 1 kHz would generally not be desirable. Indeed prior art non-ophthalmic surgeries are generally performed at very low repetition rates, typically much smaller than 1 kHz.

Even with massive FELs short pulse surgery under the condition of thermal confinement is typically very slow. For example, for an FEL operating at a fluence of 1.5 J/cm² and a pulse energy of 3 mJ at a wavelength of 6.4 μm, an ablation volume of around 0.001 mm³ per pulse has been obtained in mouse dermis (see for example Mark A. Mackanos, ‘The effect of pulse structure on soft tissue laser ablation at mid-infrared wavelengths’, Ph.D. Thesis, Vanderbilt University (2004)). Here the spot diameter was around 500 μm. This corresponds to an ablation mass of around 1 μg per pulse or an ablation efficiency η_{abl}=0.3 μg/mJ. At 10 Hz repetition rate, the ablation speed (e.g., amount of mass ablated per unit time) is only ηₐᵥ≈10 μg/s. In tooth enamel, a more recent 1 kHz repetition rate solid state laser operating at a wavelength near 3 μm with a fluence of 0.75 J/cm² has produced an ablation speed ηₐᵣᵥ≈5 μg/s and η_{abl}=0.01 μg/mJ (see, e.g., K. Franjic et al., ‘Laser selective cutting of biological tissues by impulsive heat deposition through ultrafast vibrational excitations’, Opt. Expr., vol. 17, pp. 22937 (2009). The spot diameter was around 330 μm.

Note the average laser powers deposited onto the samples for the two above examples were 0.03 W and 0.5 W respectively. Even lower average powers of around 300 μW were suggested in '674, obviously way too low for efficient ablation for most applications. In any of these cases laser ablation with short laser pulses is a slow process. For example to ablate a sphere with a diameter of 1 cm would take around 10⁵ s or around 1 day at an ablation speed of 5 μg/s.

Use of short pulses can supply laser energy under the condition of stress confinement. In the presence of some degree of stress confinement, photospallation can be exploited. Since photospallation is a photo-mechanical process it enables tissue ablation with a reduced amount of heat dissipation into the tissue. A design parameter related to the laser pulse width tₚ enabling to evaluate the significance of stress confinement is γ=tₚ/tₙ, where tₘ is the time it takes for a thermoelastic stress wave to propagate through the optical penetration depth Lₐ of the laser pulse in the tissue. When γ≦1, some benefits of stress confinement can be expected. Here tₘ=Lₐ/vₐ and vₐ is the velocity of sound in the tissue. The optical penetration depth Lₐ for various tissues is well known as a function of wavelength; for example for the water absorption peak near 2940 nm, Lₐ≈1-3 μm. Assuming the sound velocity in water of 1500 m/s we obtain tₘ≈70 ps to 2000 ps and hence the benefits of stress confinement can be obtained with pulses shorter than a few ns. Harder tissues generally have higher sound velocities and thus require shorter pulses for stress confinement. Stress can further be maximized when using pulse widths tₚ<tₘ, as described in Vogel et al., ‘Pulsed Laser Ablation of Soft Biological Tissues’ in Optical Thermal Response of Laser Irradiated Tissue (2011). Stress can further be maximized in localized areas by the presence of inhomogeneities as discussed in Franjic et al., ‘Laser selective cutting of biological tissues by impulsive heat deposition through ultrafast vibrational excitations’, Optics Express, vol. 17, pp. 22937 (2009). Indeed it has been shown that some benefits of stress confinement (or improvements in surgery performance) can already be obtained for pulse widths of a few ns, as discussed in '822.

Recently some interesting developments in beam delivery technology have taken place. A state of the art fiber based beam delivery system based on photonic crystal fibers has been discussed in A. Ulrich et al., ‘Silica hollow core microstructured fibres for mid-infrared surgical applications’, Journal of Non-Crystalline Solids, vol. 377, pp. 236-239 (2013). An energy up to ˜200 mJ could be delivered through these micro-structured fibers with a fluence of up to 2300 J/cm². However the system pulse width of 225 μs was outside the range of thermal confinement and the laser repetition rate was only 15 Hz and hence such a system would not be desirable for efficient precision surgery applications. Moreover, although high power mid-IR radiation can be transmitted by Ulrich et al., typically unwanted strong absorption windows near 2.8 μm in the fiber still pose a problem.

Photonic crystal fibers have also been shown to enable Raman shifting to frequency down-shift pulsed laser radiation, as discussed in B. Beaudou et al., ‘Matched cascade of bandgap-shift and frequency-conversion using stimulated Raman scattering in a tapered hollow-core photonic crystal fibre’, Opt. Expr., vol. 18, pp. 12381 (2010). However, no Raman shifting to the 3000 nm wavelength regime was possible and the generated energy levels were way too small for surgery applications.

Another method for generating radiation in the 3000 nm wavelength range has been based on frequency shifting of pulse bursts in an optical parametric oscillator as described in Wei et al., ‘Fiber laser pumped high power mid-infrared laser with picosecond pulse bunch output’, Opt. Expr., vol. 21, pp. 25364 (2013) and also in MIRSURG, Grant Agreement number: 224042, final report. However, the generated pulse energies were either too low for surgery applications or the system was not configured for effective precision surgery applications.

### Example Methods and Apparatus for Laser Surgery

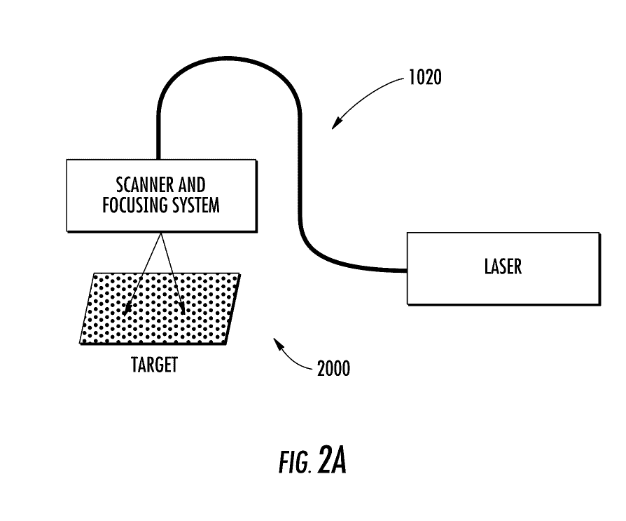

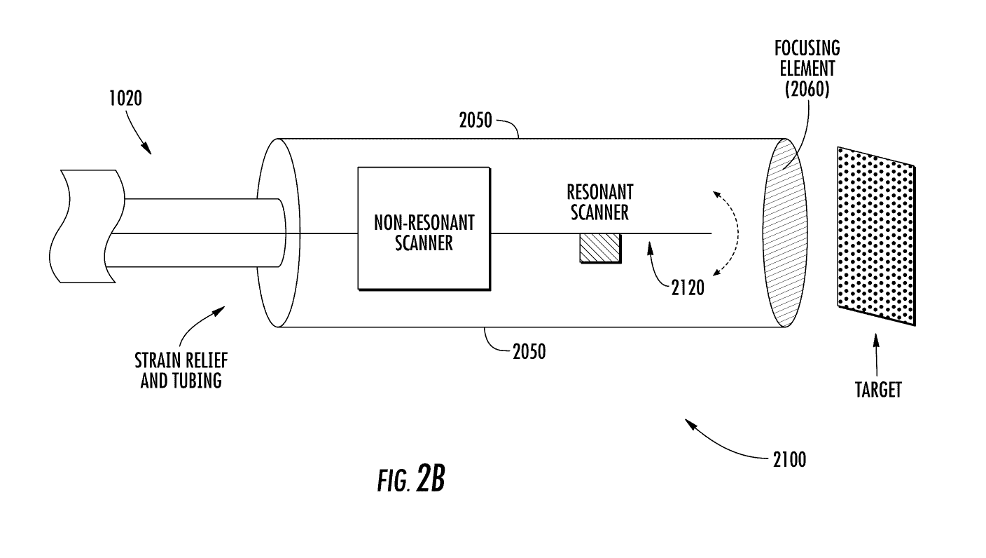

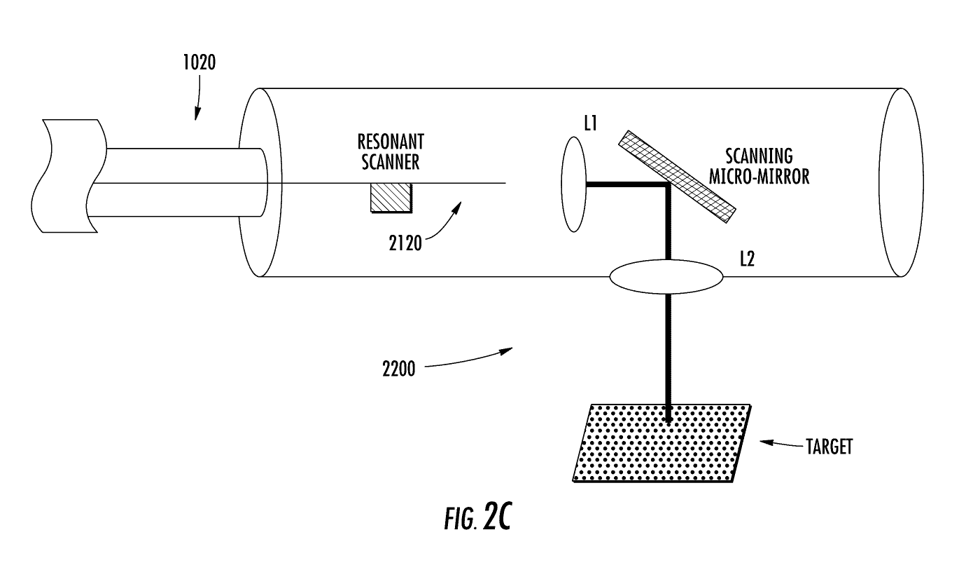

Example aspects of the present disclosure relate to methods and apparatus for laser surgery. As shown in FIG. 2A, an example apparatus 2000 includes a high repetition rate laser to perform surgery. In one implementation, the laser is delivered to the area of interest using a waveguide-based delivery system 1020. Hollow core optical fibers such as photonic crystal fibers, micro-structured fibers, hypocycloid or Kagome fibers are preferred, but any other waveguide technology capable of delivering high pulse energies can also be used. An optical scanning apparatus can be implemented to scan the laser beam over the target area in conjunction with a focusing system. The scanning system can be based on galvanometric scanners as well known in the state of the art. The scanning system can be omitted and also be included in the fiber delivery system directly or an environmental interface. The enclosure and beam delivery head may be configured with an outside diameter in the range from about 1 mm to 50 mm.

A system 2100, which includes scanning fiber delivery, is shown in FIG. 2B. The system includes a hollow waveguide or fiber 1020, which is appropriately sealed at one or both ends. For example, the fiber can be environmentally sealed by inserting at least its ends into air-tight enclosures with appropriate windows or lenses for input and output coupling. In this example the hollow core acts as a transfer fiber configured to receive said pulsed radiation from the laser source and to transfer said pulsed radiation to an output of the transfer fiber, for example a portion of fiber in the delivery system.

In an implementation, the hollow waveguide 1020 can be attached to a resonant scanner, such as a piezo-electric transducer (PZT). In this example a length of the transfer fiber proximate to the output (e.g.: fiber end 2120) is configured to resonantly vibrate in a transverse direction with respect to the fiber axis. The output fiber end provides output radiation for laser surgery. The fiber axis can be in the direction along which radiation can be propagated in the fiber (e.g., along the horizontal line that indicates the fiber end 2120). The dashed arrow in FIG. 2B shows a fiber scan path corresponding to a direction of resonant vibration of the output fiber end (e.g.: in the plane of the page of FIG. 2B, which is transverse to the fiber axis). Rapid scanning can be performed when applying a modulation frequency to the PZT in resonance with a fiber cantilever 2120. When operating the device as a resonant cantilever, the fiber tip, which may be a free, non-supported length of fiber, can be moved at speeds up to several m/s, which corresponds directly to the achievable scan rate. With magnifying optics, an equivalent magnification of the scan speed can also be achieved. For typical fiber diameters in the range from 100-300 μm and fiber cantilever lengths in the range from 5-50 mm, resonance frequencies around 1 kHz are achievable. As is well known, the resonance frequency scales with (d/L²), where d is the fiber diameter and L is the fiber cantilever length. Fiber diameters and cantilever lengths outside these ranges can also be used.

For surgery applications resonant fiber scanning in only one direction generally is sufficient. The scanning system can be sealed into an enclosure 2050 with a very small diameter to enable endoscopic surgery. Enclosure diameters between a few mm to a few cm can be used, with diameters as small as 1.0 mm being possible. To facilitate very small enclosures, the fiber diameters can also be as small as possible, for example fiber outer diameters as small as 100 μm are compatible with hollow core photonic crystal fibers. Smaller fiber outer diameters also increase the possible speed of the oscillating fiber tip, i.e. as can be shown the fiber tip speed is approximately inversely proportional to the fiber diameter.

To image the light from the fiber onto the target area a focusing element 2060 may be configured with a lens, or a general optical imaging system can be implemented. With an optical imaging system that provides an optical magnification of M, the scan speed at the target can also be magnified by a factor of M. Therefore, if large scan speeds are desired, hollow core fibers with small core diameters are a desirable option.

For more demanding applications, a non-resonant PZT scanner as shown, and an associated actuator, can be implemented as a second scanner as shown in FIG. 2B and also discussed in '622. In some embodiments a micro-mirror or MEMS-mirror can be used to scan along the second (e.g.: orthogonal) axis, such an implementation is shown in FIG. 2C. In this example, the scanning beam emerging from the fiber 2120 is collimated and focused using lenses L1 and L2, for example in a two-lens telescope configuration. Also the beam can be designed to emerge at an angle of 90 degrees with respect to the fiber axis as shown in FIG. 2C. Other incident and emergent angles can also be used.

The fiber assemblies 2100 and 2200, including the scanning devices from FIGS. 2B and 2C, can also be inserted in an appropriate enclosure as discussed above, which can be small enough to enable endoscopic beam delivery. Alternatively, all the enclosures can be adapted to enable handheld beam delivery. The enclosure can also be held at reduced gas pressure or include pressurized gas to assist with beam delivery. An appropriate gas supply is not separately shown. The input end of the enclosures can further include appropriate tubing to provide strain relief (not shown) and to direct the beam delivery system to the external laser system. Additional devices to move the output over a target area can also be included; such devices can include appropriate clamps to move the beam delivery system and to allow remote control as well known in the state of the art.

The fiber delivery system 1020 can for example include hollow-core photonic bandgap fibers, Kagome fibers, hypocycloid core or photonic crystal fibers made with alternating layers of dielectrics. These hollow-core waveguides can produce near-diffraction limited outputs and deliver pulses with peak powers of tens of MW using core diameters in the range from about 20-100 μm. Since relatively large beam diameters can be obtained from these fibers, diffraction of the beams emerging from the fibers can be relatively small and therefore, the beam can be delivered to the target area with a reasonable working distance, and in some embodiments even without any imaging or focusing optics. For example for a mode spot diameter of 90 μm, the Rayleigh length is around 2 mm at a wavelength of 3000 nm and the beam only expands to around 450 μm after a distance of 10 mm. Fibers producing near diffraction-limited output are preferably in such an operational mode. Typical hollow-core photonic bandgap fibers, Kagome fibers, hypocycloid core or photonic crystal fibers made with alternating layers of dielectrics can be configured to produce such near diffraction-limited outputs.

To reduce or minimize collateral damage during surgery the use of short pulses in the mid IR spectral region from 2.5-10.0 μm is preferred. A preferred transmission window for hollow core silica fibers is in the range from 2.6-3.8 μm. However, hollow core fibers based on other materials can also be implemented. Particularly attractive are photonic crystal fibers made from germania glass, which have much better transmission characteristics in this wavelength range compared to silica glass. Such germania glass based hollow core fibers can be manufactured via extrusion, which greatly simplifies their fabrication and cost. Residual OH contamination on the surface areas of any of such hollow core fibers can be minimized by baking the fibers at elevated temperatures, as well known in the state of the art.

In some implementations, the laser pulse width can be in the range from about 100 fs to about 10 μs. To enable spallation assisted ablation with minimal thermal damage, pulse widths in the range of about 100 ps-10 ns are preferred. However, longer pulses may be of interest for many applications. Pulse widths up to 5-10 μs can allow surgery in a thermally confined regime. Pulse widths less than 10 ps allow very small ablation spots and even sub-surface tissue modification via multi-photon excitation. When using pulse widths <10 ps, wavelengths in the range from 1.1-2.0 μm can also be efficiently implemented in some systems.

Mid-IR pulses can be generated using many different conventional laser systems or conventional lasers in conjunction with frequency conversion in nonlinear crystals as well known in the state of the art. Nonlinear frequency conversion can be accomplished with appropriate optical parametric oscillation (OPO) or optical parametric generation (OPG) and optical parametric amplification (OPA) stages as well known in the state of the art. Conventional laser systems can for example be based on Yb fiber laser technology as disclosed in '578. Also Nd or Yb based solid-state laser architectures can be used. Alternatively, Tm:fiber or Ho:fiber based laser architectures or Ho:YAG based solid-state laser architectures in conjunction with appropriate optical parametric oscillation or optical parametric generation and amplification stages can be used. Yet another alternative laser architecture can be based on Er:YAG or Er:YSGG lasers operating directly in the 2.9 μm or 2.8 μm spectral region respectively or Cr:ZnSe laser architectures operating in the 2.4-3.0 μm spectral region. The laser architectures can comprise mode locked laser front ends as well as Q-switched laser front ends. Some alternatives can also be based on gain-switched laser architectures. Other alternative can be based on temporally sliced continuous wave (cw) lasers. Temporal slicing of a short pulse form down-stream of a cw laser can for example be accomplished with a fast optical modulator. Such laser systems are well known in the state of the art and are discussed here only with respect to the following example.

An example laser system can comprise a passively or an actively Q-switched Yb:YAG micro-chip laser operating at repetition rates between 1-100 kHz and producing near bandwidth-limited pulses with pulse widths between about 100 μs-10 ns. Alternatively, other micro-chip lasers based on for example Nd:YAG or Nd:YVO₂ or Nd:YLF operating in the 1-1.07 μm wavelength range can be used. Additional amplification in a fiber amplification system based on Yb large core fibers and Yb fiber rods as well known in the state of the art can produce pulses with a peak power of a few MW. With 1 ns seed pulses output pulse energies of up to 3 mJ can be reliably obtained. After frequency conversion via OPO, OPG or OPA in a nonlinear crystal such as KTA, periodically poled LiNbO₃ or periodically poled LiTaO₃ (to name a few examples) around 500 μJ of output pulse energy at 2.9 μm can be obtained. A separate seed for injection at the signal or idler wavelength can also be included and is not separately shown. A delivery fiber as discussed above can then deliver about 250 μJ at the end of the fiber, assuming around 50% coupling and transmission losses.

To enable efficient ablation a minimum fluence of around 0.5-1 J/cm² is preferred, though other fluences may also be used. With a pulse energy of 250 μJ, such a fluence can be obtained with a spot diameter of around 180-250 μm. To operate under the conditions of thermal confinement the laser beam has to be scanned sufficiently fast to minimize pulse overlap between subsequent pulses; a rough estimate is to move the spot size by around 50% of the spot size between pulses. Assuming the laser spot size is translated by s=100 μm between subsequent pulses, the resulting scan speed is calculated as V=s×fᵣₑₚ, where fᵣₑₚ is the laser repetition rate. For a laser operating at a repetition rate of 10 kHz, we obtain V=1 m/s. Such high scan speeds can typically not be achieved by surgeons who use a conventional non-scanning handheld laser beam delivery system. As discussed above, scan speeds of 1 m/s are on the other hand not a limitation for scanning beam delivery, especially in conjunction with a magnifying optical imaging system. With sufficiently powerful lasers, ablation speeds greater than 1 mg/s are possible with scanning handheld beam delivery systems. The ablation speed can thus be more than two orders of magnitude higher than possible with FELs. Average laser powers greater than 1 W can be delivered to the target area without excessive thermal damage. Note that with a spot size of 25 μm on the target area a laser fluence of 1 J/cm² is reached with a pulse energy of only 5 μJ; for such low pulse energies, laser repetition rates greater than 10 kHz may be required for efficient tissue ablation. To avoid thermal damage arising from pulse overlap, transfer fiber producing a near diffraction-limited output are preferable. Highly multi-mode fibers would be ineffective in conjunction with a resonant fiber scanning approach, but can be used with other scanning modalities.

Rather than producing pulses at a fixed repetition rate, it can also be of advantage to use pulse bursts for surgery, as known from surgery work performed by FELs and also discussed in the MIRSURG, Grant Agreement number: 224042, final report.

The use of pulse-bursts was previously suggested to optimize laser ablation in micro-machining applications as for example described in U.S. Pat. No. 7,486,705: Temtosecond laser processing system with process parameters, controls and feedback’, L. Shah et al. (incorporated by reference herein), and U.S. Pat. No. 6,552,3101, ‘Burst-Ultrafast laser Machining Method’, P. Hermann et al. To the authors knowledge, the benefits of pulse bursts in highly efficient, high repetition rate laser surgery applications have not been reported to date.

With the use of pulse-bursts rather than individual pulses, tissue ablation can be induced with pulses with less peak power, as the pulse energy for tissue ablation can be distributed among several pulses. For example, the number of pulses in a pulse-burst can vary from 2-2000 and the pulse separation between pulses in a pulse burst can vary between twice the pulse width and about 1 μs. The pulse width can vary from fs to a few ns pulses. High repetition rate pulse trains can also produce advantageous effects through the accumulation of molecular perturbations of the tissue or other phenomena.

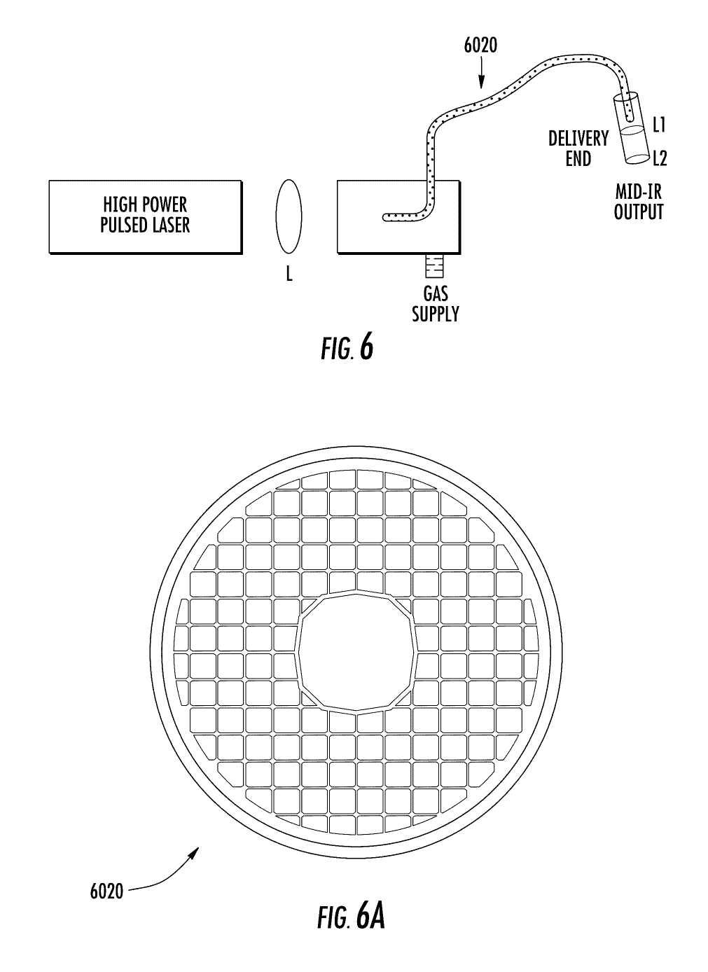

Pulse-bursts can be generated by many different methods. For example when using a fiber-based chirped pulse amplification system as described with respect to FIG. 5a in U.S. Pat. No. 7,414,780, ‘All-fiber chirped pulse amplification systems’, to Fermann et al., a pulse picker or optical modulator can be conveniently configured for the generation of pulse bursts in a power amplifier.

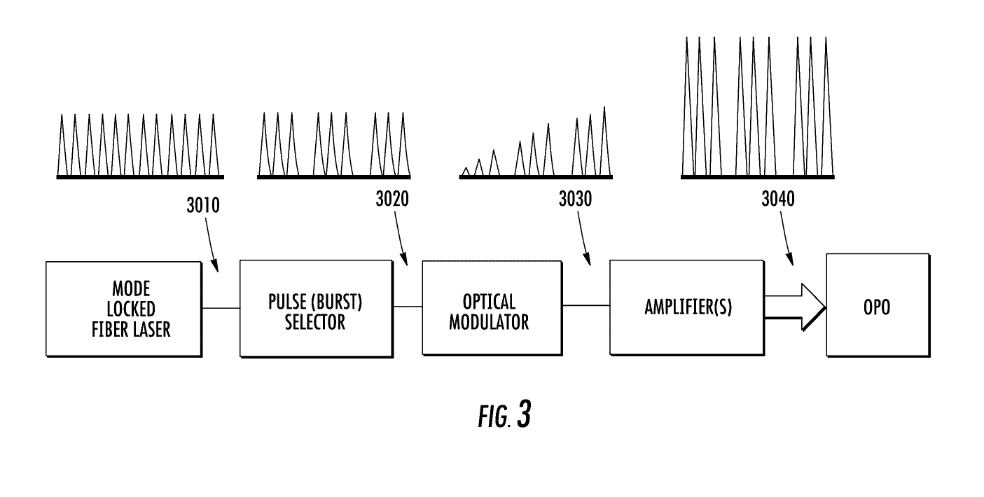

An example compact fiber laser based architecture producing pulse bursts and adapted for rapid surgery is shown in FIG. 3. For example, a robust mode locked Yb fiber laser can be used as the front end as shown in FIG. 3. Such fiber lasers can for example produce a pulse train of 10 ps-500 ps pulses at repetition rates of 100 MHz, although higher and lower repetition rates are also possible. The pulse train produces an essentially uniform train of pulses 3010. A pulse burst selector, typically configured as an acousto-optic modulator can then be programmed to select a preferred pulse pattern, such as pulse pattern 3020. The repetition rate of the pulse bursts can then be selected to be in a range of a few Hz to about 100 kHz. In a preferred implementation for high speed surgery the repetition rate may be in the range from about 1 kHz-100 kHz, about 10 kHz-100 kHz, or up to about several hundred kHz. With rapid scanning technology, laser surgery without heat accumulation can be performed even at very large repetition rates.

To generate a pulse train with uniform pulse amplitude at the output end of a final amplifier stage, amplifier saturation can be accounted for. Since amplifier saturation reduces the gain of the back end pulses within the pulse train, the front end pulses are preferably attenuated via a second optical modulator as shown in FIG. 3. This second optical modulator can comprise for example an electro-optic or acousto-optic modulator. An example pulse train 3030 is obtained at an output of the second modulator 3. The optical modulator is configured to compensate for gain saturation in a final power amplifier. Saturated amplification of the pulse burst finally generates a pulse burst with approximately uniform pulse amplitudes 3040. The approximately uniform pulse train is then injected into a synchronously pumped optical parametric oscillator. To obtain the optimum conversion efficiency of the pump light into the mid-IR in the OPO, adaptive feedback as well known in the state of the art (not shown) can further be implemented to stabilize the cavity length of the OPO. With Yb fiber amplifiers configured to amplify pulse bursts, pulse energies of 10 mJ and more can be obtained in a pulse burst. After frequency conversion in the OPO pulse energies greater than 1 mJ can be obtained in the mid IR, where mid IR average powers greater than 10 W are possible. In another embodiment, a modulator can also be used to modulate the polarization states of the pulses to for example generate pulse trains with alternating polarization states between subsequent pulses; pulse trains with alternating polarization states can for example be used to coherently combine subsequent pulses via using appropriate path delays and polarization beam splitters as well known in the state of the art.

In addition to fiber laser systems seeded with mode locked lasers, diode laser seeded fiber systems can also be implemented. In fact with gain-switched diodes, optimized pulse patterns for laser ablation can be freely selected within reasonable limitations of pulse energy, rise time, and chirping. Other laser architectures or laser media, such as laser media based on solid-state lasers can also be used to produce pulse bursts. In addition to using electronic means to produce pulse bursts, optical delay lines, pulse shapers, mechanical shutters or beam scanners as well known in the state of the art can also be implemented.

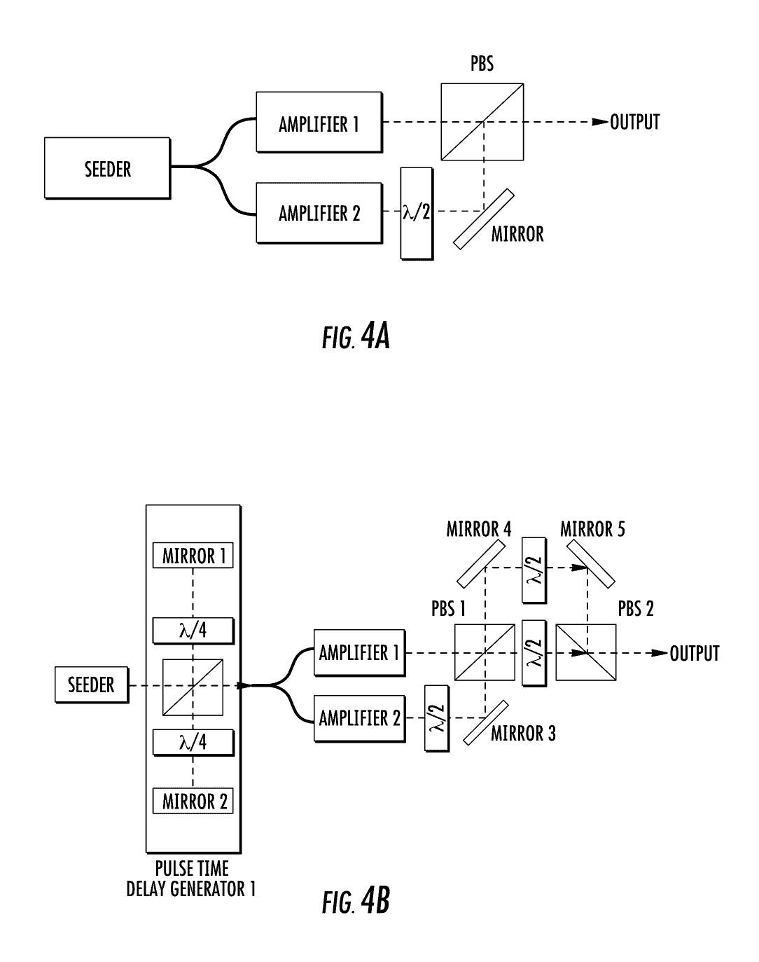

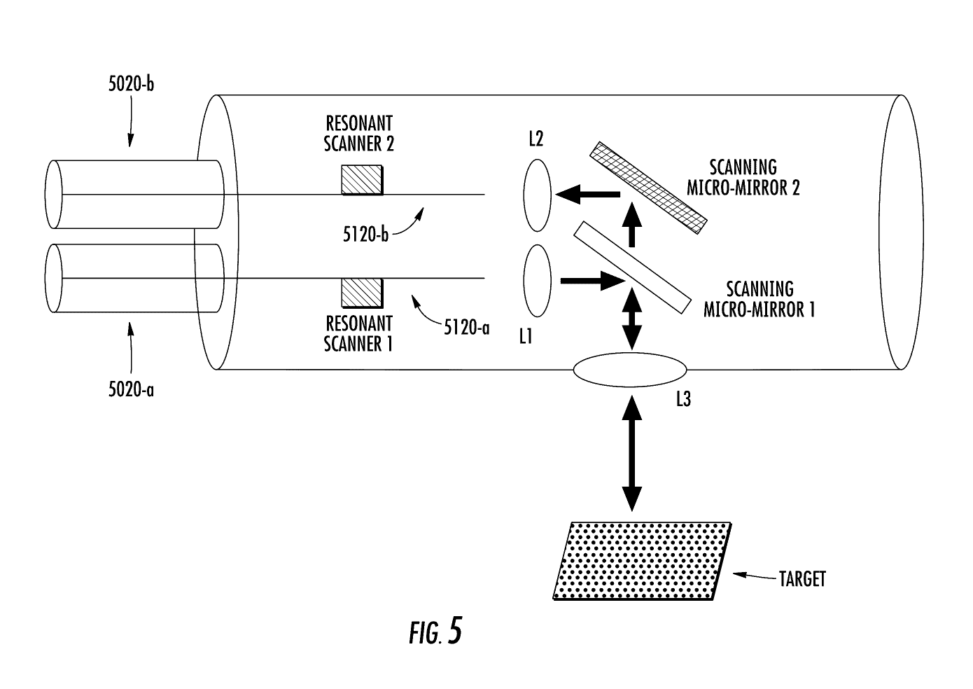

The use of pulse bursts further allows an increase in the peak pulse power by the implementation of coherent superposition or addition of at least some of the pulses in the pulse burst either before or after the frequency conversion process. To enable coherent superposition of two pulses a delay line with a differential length corresponding to the exact temporal pulse separation can be implemented. Such a system is shown in FIG. 4A. Here a seeder generates isolated individual pulses, which are then amplified in two amplifiers before being coherently combined via a polarization beam splitter PBS. A half-wave plate inserted down-stream of amplifier 2 ensures that the two polarization states impinging onto the PBS are orthogonal. To ensure coherent combination of the output of the two amplifiers, the group delay between the two pulses can be equalized and their optical phase delay can be controlled. This can be done via feedback control of the optical path length of at least one of the pulses via appropriate means such as for example control of the location of the shown mirror via an attached piezo-electric transducer PZT. Such schemes are well known in the state of the art and not further explained here.

More than two pulses can be coherently combined by superposition of for example two pairs of pulses, which are then in turn superposed (leading to the coherent combination of four pulses). An example scheme for the coherent combination of four pulses is shown in FIG. 4B. In contrast to FIG. 4A, a pulse time delay generator is inserted upstream of the two amplifiers. The pulse time delay generator generates a pulse pair with orthogonal polarization states using the arrangement with the PBS, the two quarter waveplates and two mirrors. The two pulses are further temporally separated according to the group delay in the pulse time delay generator. After amplification of the two pulse pairs in the two amplifiers, two spatially separated pulse pairs with orthogonal polarization states are generated. The four pulses are subsequently recombined into a single pulse using the arrangement with the PBS1, PBS2, mirrors 3-5 and the two half-wave plates. To ensure coherent combination of all pulses, the group delay between the pulses is equalized and the phase delay between the pulses is controlled via active feedback loops as well known in the state of the art. For example the location of mirrors 3 and 4 can be controlled via active feedback loops to control the pulse overlap. Such schemes are well known in the state of the art and not further explained here. Larger pulse numbers can also be coherently combined by further scaling of the systems shown in FIGS. 4A and 4B. Other configurations for coherent addition can also be used and are well known in the state of the art and not further discussed here.

For example instead of coherent addition with active path length control, a configuration as shown in FIG. 4B can also be used for passive coherent addition. This can for example be enabled by replacing the 50/50 fiber coupler with a PBS and replacing PBS 1 with another mirror, to configure the two amplifiers in a Sagnac loop. The Sagnac loop ensures coherent combination of the two pulse pairs after amplification in the two fiber amplifiers and directs the remaining pulse pair back to the pulse delay time generator 1. The insertion of a Faraday rotator between the pulse time delay generator and the Sagnac loop then ensures that the pulse pair is recombined into a single pulse in backpropagation through the pulse time delay generator, but with a polarization state rotated by 90 degrees. The high energy pulse can then be extracted via another Faraday rotator and a PBS upstream of the pulse time delay generator. Passive coherent combination schemes can also be upgraded to allow combination of more than four pulses. Such schemes are well known in the state of the art and not further explained here.

To perform laser surgery combining the laser delivery system with an imaging modality to evaluate the target area is to be considered. An example system configuration is shown in FIG. 5. To construct the system with an imaging modality such as optical coherence tomography (OCT), multi-photon microscopy or an endoscope, a second fiber 5120-b for imaging is added to the configuration discussed with respect to FIGS. 2A-2C. An example configuration shown in FIG. 5 can include two fibers. A first length of transfer fiber (fiber 1, 5020-a ) is connected to or integral with fiber and cantilever tip 5120-a . In this example the fiber cantilever tip 5120-a , together with resonant scanner 1, provide for optical scanning of the input beam as with the fiber and cantilever 2120 in the example system described with reference to FIGS. 2B and 2C. The fiber(s) may include a hollow core fiber for delivery of pulses with high pulse energy. A second length of transfer fiber (fiber 2, 5020-b ) is connected to or integral with fiber and cantilever tip 5120-b . Cantilever tip 5120-b , together with resonant scanner 2, provide for imaging via optical scanning, Thus, the second fiber is arranged for image acquisition. In some embodiments additional pathways can also be included for beam delivery or for collection of energy from the target tissue. In some embodiments a single fiber can be used to deliver surgery light as well as light for an imaging modality, wherein energy from the target is received in the single fiber and delivered to a photodetector (not shown). Optical beam splitters upstream of the output end of the delivery fiber can be used for beam separation. Such an example is not separately shown.

In the representative example of FIG. 5, both fibers are scanned with the two resonant PZT scanners and independent scanning mirrors, micro-mirror 1 and micro-mirror 2, and in at least one implementation the scanners have identical components and are synchronized with an external controller (not shown). Lens system L1 and L3 images the high energy beam onto the target area, whereas lens system L2 and L3 captures the scattered light from the target area and injects it into fiber 2. For OCT, fiber 2 can be a single mode fiber. For endoscopy, fiber 2 can be a multi-core fiber. To make the system compatible with multi-photon microscopy, a third fiber arm comprising a near IR laser can also be included. Fiber 2 can also be configured as a multi-mode fiber for multi-photon microscopy. Lasers with deeper penetration depths can also be used in conjunction with the surgery laser to alert a surgeon about underlying precious tissue such as nerves that is preferably not damage in the surgical procedure. Such precious tissue can for example be identified with OCT, but any other imaging modality can be used for the same purpose. Also nerve stimulation via near IR light as well known in the state of the art can be used to facilitate nerve detection.

The enclosure can be contained in a tube with a diameter of around 1-50 mm or more, depending on the surgical requirement. The system can be configured for handheld surgery, as well as for endoscopic surgery, where an endoscope is used not only for object inspection, but the laser beam is also delivered via an endoscope.

Rather than providing adaptive feedback via optical imaging modalities, other analytical tools can be provided to provide information about the target area. For example a separate suction tube can be located near the target area to suck up debris generated by the laser surgery. The debris can subsequently be transferred to a mass spectrometer for further analysis. Conveniently electro-spray ionization can be implemented to ionize desorbed molecules as may be required for analysis in a mass spectrometer. The same laser can be used for laser surgery as well as for desorption to produce a laser plume including debris at the molecular level for further analysis by the mass spectrometer. Also a separate desorption laser can be directed to the target area to increase the molecular content of the laser plume. A separate laser for ionization can also be used. The details of such a mass spectrometer and appropriate desorption lasers were for example disclosed in U.S. patent application Ser. No. 14/142,240, ‘Pulse-burst assisted electrospray ionization mass spectrometer’, filed Dec. 27, 2013, which is hereby incorporated by reference herein.

As an alternative to mass spectrometry, optical laser induced breakdown spectroscopy (LIBS) can also be used for providing adaptive feedback. To enable LIES, the light emitted at the surgery area is directed via an optical fiber to an optical spectrometer for analysis. An additional a near IR laser can also be used to enhance the LIES signal. Such modalities are compatible with the system configuration shown in FIG. 5, where for example fiber 5020-b can be used to capture and transmit the LIES signal.

In some applications, mid-IR frequency shifting in optical transfer fibers can also be considered for laser surgery. Rather than delivering high energy pulses from a laser system, the hollow core fibers are then used to generate the IR wavelengths and transfer the frequency shifted output to the target area. Such transfer fibers can for example take advantage of four-wave mixing or Raman scattering to generated the desired wavelengths.

FIG. 6 schematically illustrates a laser surgery system which incorporates an example hollow-core photonic bandgap and/or holey fiber(s) which may be implemented as transfer fiber(s), and provide for optional wavelength shifting. The system includes provisions for beam scanning as discussed with respect to FIGS. 2A - 2C. Preferably, the system uses a high power laser system operating in the 1.0-2.2 μm wavelength range at the injection end, where lens L or an imaging system is used to direct the light from the laser into the hollow core fiber 6020. Polarization control (not shown) in front of the fiber can also be used. The hollow core fiber can be based on PCFs, Kagome fibers, hypocycloid fibers or other designs. FIG. 6A schematically illustrates an end view of an example of a hollow core photonic bandgap fiber, having a square lattice, as disclosed in U.S. Pat. No. 8,478,097, ‘Wide bandwidth, low loss, photonic bandgap fibers’. Holey fiber designs may also be utilized as disclosed, for example, in U.S. Pat. No. 8,285,099, ‘Large core holey fibers’, (e.g.: FIG. 11 of '099). The entire fiber transfer system is sealed and an appropriate gas supply can be provided at the fiber input. Alternatively, provided leakage through the system is small enough, the system can be filled with a gas prior to sealing and no separate gas supply is required in some such embodiments. To minimize absorption losses near 3 μm, hollow core fibers based on for example germania glass as discussed earlier are beneficial.

Laser systems operating in the 1.0-2.2 μm can for example be based on Yb, Nd, Er, Tm, Ho or Tm:Ho fiber lasers; equally solid-state laser systems based on Nd:YAG, Yb:YAG, Ho:YAG or Tm:YAG can for example be used. Any of the laser systems can be based on mode locked laser architectures, Q-switched or gain-switched laser architectures, as well known in the state of the art. The pulse widths can be in the range from 10 μs to around 1 μs. Cw lasers temporally sliced or modulated by an external modulator and amplified in appropriate fiber or other amplifiers can also be used as an effective pulse source.

To reach the wavelength range from 1.3-3.1 μm, Raman shifting in hydrogen gas can be implemented. However, other gases with other Raman shifts can also be used. For example methane gas has a Raman shift of around 87 THz. The Raman shift in hydrogen is 17.6 THz; therefore to reach for example 2.79 μm with a second order Raman shift, a pump wavelength of 2.102 μm can be used. To reach a wavelength of 2.94 μm with a third order Raman shift, a pump wavelength of 1.937 μm can be used. Another alternative is to Raman shift a Cr:ZnSe laser operating in the 2.5 μm wavelength range to reach the 3 μm wavelength range. Typically, a hydrogen pressure of around 10 bars can be used for Raman shifting, though higher and lower pressures are also possible. With peak laser powers of a few hundred kW, efficient Raman conversion can be obtained in around a meter or a few m of hollow fiber.

Heat generated in the Raman conversion process may be a factor, however. For example, the heat generated in the conversion process may be proportional to the changes in Stokes intensity along the fiber length and the quantum defect between the pump and the Raman photon. Hence most of the heat is generated after an initial build up length of the Raman signal. At the beginning and end of the Raman shifting fiber the heat generated can be minimal. Therefore heat generation can be effectively managed by cooling the middle section of the fiber via heat-sinking or water cooling while leaving the fiber ends unobstructed.

The thermal conductivity of H₂ gas at 10 atmospheres (atm) is around 6.3 times smaller compared to the thermal conductivity of silica glass and comparable to the thermal conductivity of typical fiber polymer coatings. For a typical Kagome or hypocycloid fiber it is therefore efficient to fill the fiber gaps with the Raman gas (such as H₂) rather than vacuum to enable efficient heat dissipation. Heat generation can then be modelled assuming a confined heat source (in the fiber core) in a medium with uniform thermal conductivity. Such calculations are well known from the analysis of heat flow in conventional fiber lasers. Indeed, we have verified that with appropriate heat sinking of a hollow Raman shifting fiber for a heat load of around 30 W/m the temperature increase inside the fiber core can be limited to around 100° C., whereas without any cooling, the temperature of the H₂ gas could exceed 1000 ° C. Hence efficient fiber cooling can allow the generation of >100 mW of power using a length of a few m of hollow core fiber. Even power levels >1 W and >10 W are possible near the 3 μm spectral range.

Heat can be more efficiently dissipated the longer the fiber length. Also, the fiber can be tapered along the length to optimize the conversion efficiency. Frequency conversion efficiencies significantly higher than 10% are possible. To avoid unwanted absorptive losses of the pump or Raman signal, absorption losses in the hollow core fiber for example due to well tabulated quadrupole transitions in H₂ are preferably avoided. This can for example be accomplished by using a tunable narrow band pump wavelength outside of any absorption bands. Also, a reduction of Raman gas pressure to limit pressure broadening in the Raman gas can be beneficial; for example pressure broadening of absorption lines due to H₂ gas at 10 atm is around 1 nm at 2 μm, leading to significant overlap of adjacent H₂ absorption bands near 2 μm. Simply a reduction of pressure to 5 atm reduces pressure broadening by a factor of two and provides much broader transmission windows through the H₂ gas. Pump lasers with a spectral bandwidth <1 nm are moreover preferred to limit absorptive losses for this purpose. Similar considerations also apply to other gases. OH absorption due to surface contamination is also a concern, but can be minimized by baking the hollow fiber at an elevated temperature as well known in the state of the art.

To avoid the heat generated via stimulated Raman scattering, four-wave mixing can be implemented. To enable four-wave mixing a single high energy short pulse can be injected into a fiber at a wavelength different from the zero dispersion wavelength. Phase matched four-wave mixing can then be obtained when the phase-matching condition is fulfilled, e.g., 2kₚᵤₘₚ=k_{signal}+k_{idler}+γP, where kₚᵤₘₚ, k_{signal} and k_{idler} correspond to the propagation constants at the pump, signal and idler wavelengths and γP is the nonlinear contribution to the phase matching condition dependent on pump power P and the nonlinearity parameter γ of the fiber. Both solid as well as hollow core fibers can be used for four-wave mixing. Gas filled fibers further allow a manipulation of the phase matching condition via an adjustment of gas pressure.

For efficient four-wave mixing, the injection of a second laser beam into the transfer fiber can also be used. For example four-wave mixing between two lasers operation at 1.56 μm and 2.05 μm can generate an output near 3.0 μm provided appropriate phase matching is ensured.

The systems discussed above are essentially compatible with any precision surgical instruments as well as robotic surgery. In addition the laser architectures can also be used in machining applications, laser deposition of polymers, as well as sources for laser desorption in conjunction with mass spectrometry.

### ADDITIONAL EXAMPLES AND EMBODIMENTS

In a 1st aspect, a laser surgery apparatus comprises a high repetition rate laser pulse source configured to generate pulsed radiation; and a transfer fiber configured to receive said pulsed radiation from said source and to transfer said pulsed radiation along a fiber axis to an output of said transfer fiber, wherein a length of said transfer fiber proximate to said output is configured to resonantly vibrate in a transverse direction with respect to the fiber axis so as to deliver said pulsed radiation for laser surgery.

In a 2nd aspect, the laser surgery apparatus according to aspect 1, wherein said repetition rate is greater than about 1 kHz.

In a 3rd aspect, the laser surgery apparatus according to aspect 1 or aspect 2, wherein said repetition rate is greater than about 10 kHz.

In a 4th aspect, the laser surgery apparatus according to any one of aspects 1-3, wherein the transfer fiber comprises a hollow fiber selected from one or more of: photonic crystal fiber, Kagome fiber, or a hypocycloid fiber.

In a 5th aspect, the laser surgery apparatus according to aspect 4, said hollow fiber comprising germania glass.

In a 6th aspect, the laser surgery apparatus according to aspect 5, wherein said hollow fiber is manufactured via extrusion.

In a 7th aspect, the laser surgery apparatus according to any one of aspects 1-6, wherein said transfer fiber comprises hollow fiber optimized for transmission in the approximate 1.8 μm to 3.5 μm wavelength range.

In an 8th aspect, the laser surgery apparatus according to any one of aspects 1-7, wherein said pulse source is configured to deliver high energy pulses in the wavelength range from about 1.1 μm to about 3.5 μm.

In a 9th aspect, the laser surgery apparatus according to any one of aspects 1-8, wherein said pulse source produces pulses with a width in the range from about 100 fs to about 10 s.

In a 10th aspect, the laser surgery apparatus according to any one of aspects 1-9, wherein said pulse source produces one or more pulses with a pulse width within the thermal confinement time of a target area.

In an 11th aspect, the laser surgery apparatus according to any one of aspects 1-10, wherein said pulse source produces one or more pulses with a pulse width within the stress confinement time of a target area.

In a 12th aspect, the laser surgery apparatus according to any one of aspects 1-11, wherein said pulse source comprises a gain fiber, a semiconductor diode, solid-state laser system, or a combination thereof.

In a 13th aspect, the laser surgery apparatus according to any one of aspects 1-12, wherein said pulse source further comprises a fiber amplifier system and a micro-chip seed laser.

In a 14th aspect, the laser surgery apparatus according to any one of aspects 1-13, wherein said pulse source further comprises a fiber amplifier system and a fiber based seed laser.

In a 15th aspect, the laser surgery apparatus according to any one of aspects 1-14, further comprising at least one frequency converter.

In a 16th aspect, the laser surgery apparatus according to aspect 15, wherein said frequency converter comprises at least one of or a combination of an OPO, OPA or OPG.

In a 17th aspect, the laser surgery apparatus according to any one of aspects 1-16, wherein said pulse source is configured to produce a burst of pulses.

In an 18th aspect, the laser surgery apparatus according to aspect 17, wherein said burst of pulses is derived from a mode locked fiber laser in conjunction with a down-counter.

In a 19th aspect, the laser surgery apparatus according to aspect 17 or aspect 18, wherein said burst of pulses is amplitude modulated with an optical modulator to compensate for gain saturation in a final power amplifier disposed downstream from said optical modulator.

In a 20th aspect, the laser surgery apparatus according to any one of aspects 17-19, wherein said burst of pulses is polarization modulated with an optical modulator to generate pulses with varying polarization states downstream of said modulator.

In a 21st aspect, the laser surgery apparatus according to aspect 20, further comprising at least one delay line to coherently add at least two pulses from said burst of pulses.

In a 22nd aspect, the laser surgery apparatus according to any one of aspects 1-21, further comprising an imaging system to image said output of said transfer fiber onto a target area.

In a 23rd aspect, the laser surgery apparatus according to any one of aspects 1-22, further comprising: a positioning modality having an additional actuator for non-resonant movement of said transfer fiber at a rate slower than the resonant vibrations of said fiber output.

In a 24th aspect, the laser surgery apparatus according to any one of aspects 1-23, further comprising a positioning modality having an additional actuator for moving the beam emerging from said transfer fiber along the target area.

In a 25th aspect, the laser surgery apparatus according to any one of aspects 1-24, wherein said apparatus is configured as a laser endoscope for transferring a high power laser beam to a human body cavity.

In a 26th aspect, the laser surgery apparatus according to aspect 25, wherein said apparatus is configured with a beam delivery head with a diameter between 1 to 50 mm.

In a 27th aspect, the laser surgery apparatus according to any one of aspects 1-26, wherein said apparatus is configured with a handheld beam pointer interface.

In a 28th aspect, the laser surgery apparatus according to any one of aspects 1-27, further comprising a visible beam pointing beam.

In a 29th aspect, the laser surgery apparatus according to any one of aspects 1-28, further comprising a laser beam for photo-coagulation.

In a 30th aspect, the laser surgery apparatus according to any one of aspects 1-29, further comprising at least one additional signal fiber configured to receive feedback from the laser surgery target area in form of optical signals.

In a 31st aspect, the laser surgery apparatus according to aspect 30, said at least one additional signal fiber comprising a single-mode fiber or a multi-mode or multi-core fiber.

In a 32nd aspect, the laser surgery apparatus according to aspect 30 or aspect 31, said optical signals being used for one or a combination of OCT, multi-photon microscopy, optical imaging, mid-IR imaging, or thermal imaging.

In a 33rd aspect, the laser surgery apparatus according to any one of aspects 1-32, wherein said transfer fiber provides a nearly diffraction limited output beam.

In a 34th aspect, a laser surgery apparatus comprising a high repetition rate laser pulse source operating at a repetition rate greater than about 1 kHz, wherein said pulse source is configured to generate pulsed radiation in the spectral range from about 1.1 μm to about 3.5 μm with a pulse energy greater than about 5 μJ; and a transfer fiber configured to receive said pulsed radiation from said source and to transfer said pulse radiation to an output of said transfer fiber, wherein said laser surgery apparatus is configured to emit said pulse radiation from said output of said transfer fiber during scanning over a tissue target area.

In a 35th aspect, the laser surgery apparatus according to aspect 34, wherein said transfer fiber output is near diffraction limited.

In a 36th aspect, a laser surgery apparatus comprising a laser pulse source configured to generate pulsed radiation; a transfer fiber configured to receive said pulsed radiation; and a frequency converter configured to shift a wavelength of said pulsed radiation to a wavelength for laser surgery, said frequency converter disposed upstream of an output of said transfer fiber, said frequency shifted radiation being transferred with said transfer fiber for laser surgery.

In a 37th aspect, the laser surgery apparatus according to aspect 36, wherein said transfer fiber is configured for frequency shifting via stimulated Raman scattering.

In a 38th aspect, the laser surgery apparatus according to aspect 36 or aspect 37, wherein said transfer fiber is configured for frequency shifting via Four Wave Mixing.

In a 39th aspect, a method for laser surgery comprising generating high repetition rate pulsed radiation; transferring said pulsed radiation along a fiber axis of a transfer fiber to an output of said transfer fiber; and resonantly vibrating a length of said transfer fiber proximate to said output in a transverse direction with respect to the fiber axis so as to deliver said pulsed radiation for laser surgery.

In a 40th aspect, the method of aspect 39, wherein generating the high repetition rate pulsed radiation comprises generating the pulsed radiation at a repetition rate greater than about 1 kHz.

In a 41st aspect, the method of aspect 39 or aspect 40, wherein generating the high repetition rate pulsed radiation comprises generating the pulsed radiation in the spectral range from about 1.7 to about 3.5 μm.

In a 42nd aspect, the method of any one of aspects 39-41, wherein generating the high repetition rate pulsed radiation comprises generating the pulsed radiation with at least some pulses having a pulse energy greater than about 5 μJ.

In a 43rd aspect, the method of any one of aspects 39-42, further comprising shifting a wavelength of said pulsed radiation to a wavelength for laser surgery.

### CONCLUSION

It is to be understood that the embodiments described herein are not mutually exclusive, and elements described in connection with one embodiment may be combined with, or eliminated from, other embodiments in suitable ways to accomplish desired design objectives.

For purposes of summarizing the present disclosure, certain aspects, advantages, examples, and novel features of the present disclosure are described herein. It is to be understood, however, that not necessarily all such advantages may be achieved in accordance with any particular embodiment. Thus, the present disclosure may be embodied or carried out in various manners that achieve one or more advantages without necessarily achieving other advantages as may be taught or suggested herein. No feature or group of features is necessary or indispensable for each embodiment.

The example experiments, experimental data, tables, graphs, plots, photographs, figures, and processing and/or operating parameters (e.g., values and/or ranges) described herein are intended to be illustrative of operating conditions of the disclosed systems and methods and are not intended to limit the scope of the operating conditions for various embodiments of the methods and systems disclosed herein. Additionally, the experiments, experimental data, calculated data, tables, graphs, plots, photographs, figures, and other data disclosed herein demonstrate various regimes in which embodiments of the disclosed systems and methods may operate effectively to produce one or more desired results. Such operating regimes and desired results are not limited solely to specific values of operating parameters, conditions, or results shown, for example, in a table, graph, plot, figure, or photograph, but also include suitable ranges including or spanning these specific values. Accordingly, the values disclosed herein include the range of values between any of the values listed or shown in the tables, graphs, plots, figures, photographs, etc. Additionally, the values disclosed herein include the range of values above or below any of the values listed or shown in the tables, graphs, plots, figures, photographs, etc. as might be demonstrated by other values listed or shown in the tables, graphs, plots, figures, photographs, etc. Also, although the data disclosed herein may establish one or more effective operating ranges and/or one or more desired results for certain embodiments, it is to be understood that not every embodiment need be operable in each such operating range or need produce each such desired result. Further, other embodiments of the disclosed systems and methods may operate in other operating regimes and/or produce other results than shown and described with reference to the example experiments, experimental data, tables, graphs, plots, photographs, figures, and other data herein.

Other systems, setups, and parameters may be used in other implementations, which may provide the same or different results. Many variations are possible and are contemplated within the scope of this disclosure. Materials, components, features, structures, and/or elements may be added, removed, combined, or rearranged. Additionally, process or method steps may be added, removed, or reordered. No single feature or step, or group of features or steps, is indispensable or required for each embodiment.

Certain processing steps or acts of the methods disclosed herein may be implemented in hardware, software, or firmware, which may be executed by one or more general and/or special purpose computers, processors, or controllers, including one or more floating point gate arrays (FPGAs), programmable logic devices (PLDs), application specific integrated circuits (ASICs), and/or any other suitable processing device. In certain embodiments, one or more functions provided by a controller or a control means may be implemented as software, instructions, logic, and/or modules executable by one or more hardware processing devices. In some embodiments, the software, instructions, logic, and/or modules may be stored on computer-readable media including non-transitory storage media implemented on a physical storage device and/or communication media that facilitates transfer of information. In various embodiments, some or all of the steps or acts of the disclosed methods or controller functionality may be performed automatically by one or more processing devices. Many variations are possible.

The term “or” is used in this application its inclusive sense (and not in its exclusive sense), unless otherwise specified. In addition, the articles “a” and “an” as used in this application and the appended claims are to be construed to mean “one or more” or “at least one” unless specified otherwise

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. As used herein, a phrase referring to “at least one of a list of items refers to any combination of those items, including single members. As an example, “at least one of: a, b, or c” is intended to cover: a, b, c, a-b, a-c, b-c, and a-b-c.

Thus, while only certain embodiments have been specifically described herein, it will be apparent that numerous modifications may be made thereto without departing from the spirit and scope of the disclosure. Further, acronyms are used merely to enhance the readability of the specification and claims. It should be noted that these acronyms are not intended to lessen the generality of the terms used and they should not be construed to restrict the scope of the claims to the embodiments described therein.

What is claimed is:

1. A laser surgery apparatus comprising:
a laser pulse source configured to generate pulsed radiation at a high repetition rate; and
a transfer fiber configured to receive said pulsed radiation from said laser pulse source and to transfer said pulsed radiation along a fiber axis to an output of said transfer fiber,
wherein a length of said transfer fiber proximate to said output is configured to resonantly vibrate in a transverse direction with respect to the fiber axis so as to deliver said pulsed radiation for laser surgery.

2. The laser surgery apparatus according to claim 1, wherein the transfer fiber comprises a hollow fiber selected from one or more of: a photonic crystal fiber, a Kagome fiber, or a hypocycloid fiber.

3. The laser surgery apparatus according to claim 2, wherein said hollow fiber comprises germania glass.

4. The laser surgery apparatus according to claim 1, wherein said transfer fiber comprises hollow fiber optimized for transmission in the approximate 1.0 μm to 3.5 μm wavelength range.

5. The laser surgery apparatus according to claim 1, wherein said laser pulse source is configured to deliver high energy pulses in a wavelength range from about 1.0 μm to about 3.5 μm.

6. The laser surgery apparatus according to claim 1, wherein said laser pulse source comprises a gain fiber, a semiconductor diode, a solid-state laser system, or a combination thereof.

7. The laser surgery apparatus according to claim 1, wherein said laser pulse source comprises a fiber amplifier system and a micro-chip seed laser.

8. The laser surgery apparatus according to claim 1, wherein said laser pulse source comprises a fiber amplifier system and a fiber based seed laser.

9. The laser surgery apparatus according to claim 1, further comprising at least one frequency converter.

10. The laser surgery apparatus according to claim 9, wherein said at least one frequency converter comprises at least one of or a combination of an OPO stage, an OPA stage, or an OPG stage.

11. The laser surgery apparatus according to claim 1, wherein said laser pulse source is configured to produce a burst of pulses.

12. The laser surgery apparatus according to claim 11, wherein said burst of pulses is polarization modulated with an optical modulator to generate pulses with varying polarization states downstream of said modulator.

13. The laser surgery apparatus according to claim 12, further comprising at least one delay line to coherently add at least two pulses from said burst of pulses.

14. The laser surgery apparatus according to claim 1, further comprising an imaging system to image said output of said transfer fiber onto a target area.

15. The laser surgery apparatus according to claim 1, further comprising: a positioning system having an additional actuator for non-resonant movement of said transfer fiber at a rate slower than the resonant vibrations of said fiber output.

16. The laser surgery apparatus according to claim 1, further comprising a positioning system having an additional actuator for moving the beam emerging from said transfer fiber along the target area.

17. The laser surgery apparatus according to claim 1, further comprising a laser beam device configured to output a laser beam for photo-coagulation.

18. The laser surgery apparatus according to claim 1, further comprising at least one additional signal fiber configured to receive feedback from the laser surgery target area in form of optical signals.

19. The laser surgery apparatus according to claim 18, wherein said optical signals are used for one or a combination of OCT, multi-photon microscopy, optical imaging, mid-IR imaging, or thermal imaging.

20. The laser surgery apparatus according to claim 1, wherein said transfer fiber is configured to provide a nearly diffraction limited output beam.

21. A laser surgery apparatus comprising:
a high repetition rate laser pulse source configured to operate at a repetition rate greater than about 1 kHz, wherein said laser pulse source is configured to generate pulsed radiation in a spectral range from about 1.1 μm to about 3.5 μm with a pulse energy greater than about 5 μJ; and
a transfer fiber configured to receive said pulsed radiation from said laser pulse source and to transfer said pulsed radiation to an output of said transfer fiber,
wherein said laser surgery apparatus is configured to emit said pulsed radiation from said output of said transfer fiber during scanning over a tissue target area.

22. A laser surgery apparatus comprising:
a laser pulse source configured to generate pulsed radiation;
a transfer fiber configured to receive said pulsed radiation; and
a frequency converter configured to shift a wavelength of said pulsed radiation to a wavelength for laser surgery, said frequency converter disposed upstream of an output of said transfer fiber, said frequency shifted radiation being transferred with said transfer fiber for laser surgery.

23. A laser surgery apparatus according to claim 22, wherein said transfer fiber is configured for frequency shifting via stimulated Raman scattering.

24. A laser surgery apparatus according to claim 23, wherein said transfer fiber is configured for frequency shifting via Four Wave Mixing.

25. A method for laser surgery comprising:
generating high repetition rate pulsed radiation;
transferring said pulsed radiation along a fiber axis of a transfer fiber to an output of said transfer fiber; and
resonantly vibrating a length of said transfer fiber proximate to said output in a transverse direction with respect to the fiber axis of the transfer fiber so as to deliver said pulsed radiation for laser surgery.

26. The method of claim 25, wherein generating the high repetition rate pulsed radiation comprises generating the pulsed radiation with at least some pulses having a pulse energy greater than about 5 μJ.
